(19) United States
(12) Patent Application Publication
(10) Pub. No.: US 2026/0158088 A1
(43) Pub. Date: Jun. 11, 2026

(54) COMPOSITION FOR CURING HANGOVER OR IMPROVING LIVER FUNCTION, COMPRISING PINE HEARTWOOD EXTRACT AS ACTIVE INGREDIENT

(71) Applicants: KOREA INSTITUTE OF SCIENCE AND TECHNOLOGY, Seoul (KR); DONGGUK UNIVERSITY INDUSTRY-ACADEMIC COOPERATION FOUNDATION, Seoul (KR)

(72) Inventors: Jin Chul KIM, Gangneung-si (KR); Jae Young CHOI, Gangneung-si (KR); Eun Ha LEE, Gangneung-si (KR); Sung Chul HONG, Gangneung-si (KR); Seung Hoon YANG, Seoul (KR); A Hyeon LEE, Nonsan-si (KR); Hyo Ryeong SONG, Daegu (KR)

(73) Assignees: KOREA INSTITUTE OF SCIENCE AND TECHNOLOGY, Seoul (KR); DONGGUK UNIVERSITY INDUSTRY-ACADEMIC COOPERATION FOUNDATION, Seoul (KR)

(21) Appl. No.: 18/707,936

(22) PCT Filed: Nov. 7, 2022
(86) PCT No.: PCT/KR2022/017320

§ 371 Date: May 7, 2024

(30) Foreign Application Priority Data
Nov. 9, 2021 (KR) 10-2021-0153239

(51) Int. Cl. A61K 36/15; A61K 9/00; A61P 43/00

(52) U.S. Cl. CPC A61K 36/15; A61K 9/0053; A61P 43/00; A61K 2236/331; A61K 2236/333

(57) ABSTRACT

The present invention provides a composition for curing a hangover or preventing, alleviating or treating alcoholic liver disease, comprising a pine heartwood extract as an active ingredient.

### TECHNICAL FIELD

The present disclosure relates to a composition for curing a hangover or improving liver function containing a pine heartwood extract as an active ingredient.

### BACKGROUND ART

Korea's alcohol consumption is the highest in Asia, and the annual alcohol consumption per capita in 2017 (8.7 L) is similar to the OECD average (8.9 L), ranking 13^{th} out of 36 OECD member countries (source: WHO 2019). In Korea, consumption of distilled liquor (soju) with high alcohol content accounts for 81% of all alcohol, which is known to have a significant impact on occurrence of alcoholic liver disease. Many people suffer from hangover symptoms (thirst, general malaise, fatigue, memory loss, abdominal distension, indigestion, vomiting, diarrhea, etc.) caused by excessive alcohol intake, and alcoholic liver disease caused by continuous drinking accounts for about 15% of all liver disease and primarily progresses to alcoholic fatty liver disease, of which 10-35% progresses to alcoholic hepatitis and 10-20% progresses to alcoholic cirrhosis, and about 40% of alcoholic hepatitis develops into alcoholic cirrhosis.

Generally, alcohol is broken down in small amounts in the stomach, and alcohol absorbed into the small intestine through the stomach moves to the liver through blood vessels. The liver is the most important organ in alcohol decomposition. 90% or more of ingested alcohol is broken down in the liver, and 2-5% of ingested alcohol is not broken down and is excreted through urine, sweat, and breathing. Absorbed alcohol is metabolized in the liver and mainly turns into acetaldehyde through two pathways. Alcohol is mainly broken down into acetaldehyde by ADH (alcohol dehydrogenase), but when alcohol concentration in the body increases due to excessive drinking, MEOS (microsomal ethanol oxidizing system) is activated for alcohol treatment. Acetaldehyde, which is a primary metabolite essential in such a metabolic process, causes hangovers, induces accumulation of fatigue substances such as lactic acid, and reduces the amount of vitamins in the blood by inhibiting vitamin activation, and also, large amounts of water and various electrolytes are released outside the body through urine and sweat, causing thirst, headache, lethargy, etc. In the long run, alcoholic fatty liver disease or alcoholic hepatitis may be induced, or cirrhosis may occur upon continuous drinking in a state in which the liver is damaged. Acetaldehyde is metabolized into non-toxic acetic acid by ADLH (acetaldehyde dehydrogenase), and acetic acid is converted into acetyl-CoA through another metabolic process, which is then used for energy synthesis. Acetaldehyde dehydrogenase includes type II, which acts when acetaldehyde is in low concentration, and type I, which acts only when acetaldehyde is in high concentration. Compared to other races, Asians are deficient or lacking in acetaldehyde dehydrogenase type II, so the decomposition of acetaldehyde is slow. Hence, various hangover symptoms are often felt due to acetaldehyde that accumulates in the body without being broken down.

By the nature of Korea's drinking culture, many people have trouble leading a normal life the next day due to hangovers from frequent and heavy drinking, which causes social and economic damage due to reduced work efficiency. To prevent this, sales in the hangover cure market have been steadily increasing. Due to consumer needs, thorough research and experimentation are ongoing in various fields to relieve hangovers caused by drinking, but since objective verification of the efficacy of most final products is insufficient, overall reliability of the products is low.

Accordingly, there is an urgent need for development of materials that may help lead a healthy life by objectively verifying hangover cure efficacy, building consumer trust, and reducing social losses caused by excessive drinking.

Meanwhile, pine (Pinus densiflora) is an evergreen coniferous tree that grows widely in forests throughout Asia, including Korea, China, Japan, etc. Pine leaves are known to be effective at treating high blood pressure, arteriosclerosis, headache, toothache, edema, bruises, insomnia, blood-tinged sputum, and fatigue recovery.

However, there is little development of products for curing hangovers using pine, especially the heartwood of pine.

### DISCLOSURE

### Technical Problem

In order to solve the above problems, the present inventors have searched for compositions for curing hangovers and for ameliorating alcoholic liver disease using natural materials, and ascertained the efficacy of a pine heartwood extract on ameliorating hepatotoxicity caused by acute alcohol when administered, thus culminating in the present disclosure.

Accordingly, the present disclosure is intended to provide a composition for curing a hangover and a pharmaceutical composition for ameliorating, preventing, or treating alcoholic liver disease, including a pine heartwood extract as an active ingredient.

The objects of the present disclosure are not limited to the foregoing. The objects of the present disclosure will be able to be clearly understood through the following description and to be realized by the means described in the claims and combinations thereof.

### Technical Solution

In order to accomplish the above object, the present disclosure provides the following composition.

An aspect of the present disclosure provides a composition for curing a hangover, including a pine heartwood extract as an active ingredient.

In the composition according to an aspect of the present disclosure, the pine heartwood extract may be an extract of water, C₁-C₆ alcohol, or an aqueous C₁-C₆ alcohol solution.

In the composition according to an aspect of the present disclosure, the concentration of the aqueous C₁-C₆ alcohol solution may be 10% to 98% (v/v).

In the composition according to an aspect of the present disclosure, the active ingredient may promote activity of alcohol dehydrogenase (ADH).

In the composition according to an aspect of the present disclosure, the active ingredient may promote activity of acetaldehyde dehydrogenase (ALDH).

Another aspect of the present disclosure provides a composition for preventing, ameliorating, or treating alcoholic liver disease, including a pine heartwood extract as an active ingredient.

In the composition according to another aspect of the present disclosure, the pine heartwood extract may be an extract of water, C₁-C₆ alcohol, or an aqueous C₁-C₆ alcohol solution.

In the composition according to another aspect of the present disclosure, the concentration of the aqueous C₁-C₆ alcohol solution may be 10% to 98% (v/v).

In the composition according to another aspect of the present disclosure, the active ingredient may promote activity of alcohol dehydrogenase (ADH).

In the composition according to another aspect of the present disclosure, the active ingredient may promote activity of acetaldehyde dehydrogenase (ALDH).

In another aspect of the present disclosure, the composition may be a functional health food.

In another aspect of the present disclosure, the composition may be a pharmaceutical composition.

### Advantageous Effects

A composition of the present disclosure can be used as a composition for curing a hangover. In addition, the composition of the present disclosure can be used to prevent, alleviate, or treat alcoholic liver disease.

The effects of the present disclosure are not limited to the foregoing. It should be understood that the effects of the present disclosure include all effects that can be inferred from the following description.

### DESCRIPTION OF DRAWINGS

FIG. 1A shows changes in the amount of ethanol in mouse blood 1 hour after treatment according to Example 2-3 of the present disclosure;

FIG. 1B shows changes in the amount of ethanol in mouse blood 3 hours after treatment according to Example 2-3 of the present disclosure;

FIG. 1C shows changes in the amount of ethanol in mouse blood 5 hours after treatment according to Example 2-3 of the present disclosure;

FIG. 2A shows results of an alcohol control group not treated with an active ingredient according to Example 2-4 of the present disclosure;

FIG. 2B shows results of a pine needle water extract according to Example 2-4 of the present disclosure.

FIG. 2C shows results of a pine needle ethanol extract according to Example 2-4 of the present disclosure;

FIG. 2D shows results of a Pinus densiflora root water extract according to Example 2-4 of the present disclosure;

FIG. 2E shows results of a Pinus densiflora root ethanol extract according to Example 2-4 of the present disclosure;

FIG. 2F shows results of a heartwood ethanol extract according to Example 2-4 of the present disclosure;

FIG. 3A shows changes in the activity of aldehyde dehydrogenase in mice 1 hour after treatment according to Example 2-5 of the present disclosure;

FIG. 3B shows changes in the activity of aldehyde dehydrogenase in mice 3 hours after treatment according to Example 2-5 of the present disclosure; and

FIG. 3C shows changes in the activity of aldehyde dehydrogenase in mice 5 hours after treatment according to Example 2-5 of the present disclosure.

### [Best Mode]

Unless otherwise specified, all numbers, values, and/or representations that express the amounts of components, reaction conditions, and compositions used herein are to be taken as approximations including various uncertainties affecting measurement that inherently occur in obtaining these values, among others, and thus should be understood to be modified by the term “about” in all cases. Furthermore, when a numerical range is disclosed in this specification, the range is continuous, and includes all values from the minimum value of said range to the maximum value thereof, unless otherwise indicated. Moreover, when such a range pertains to integer values, all integers including the minimum value to the maximum value are included, unless otherwise indicated.

In the present specification, when a range is described for a variable, it will be understood that the variable includes all values including the end points described within the stated range. For example, the range of “5 to 10” will be understood to include any subranges, such as 6 to 10, 7 to 10, 6 to 9, 7 to 9, and the like, as well as individual values of 5, 6, 7, 8, 9 and 10, and will also be understood to include any value between valid integers within the stated range, such as 5.5, 6.5, 7.5, 5.5 to 8.5, 6.5 to 9, and the like. Also, for example, the range of “10% to 30%” will be understood to include subranges, such as 10% to 15%, 12% to 18%, 20% to 30%, etc., as well as all integers including values of 10%, 11%, 12%, 13% and the like up to 30%, and will also be understood to include any value between valid integers within the stated range, such as 10.5%, 15.5%, 25.5%, and the like.

Hereinafter, a detailed description will be given of the present disclosure.

An aspect of the present disclosure provides a composition for curing a hangover, including a pine heartwood extract as an active ingredient.

In an aspect of the present disclosure, examples of pine may include Pinus densiflora Sieb & Zucc., Pinus thumbergii Parlatore, Pinus densiflora Sieb & Zucc. For. erecta Uyeki, Pinus sylvestris. L., Pinus pinea Cupressus Sempervirens, etc.

The heartwood of pine is distinct from the roots and leaves thereof and refers to the portion contained within the trunk of pine. More specifically, a pine log may be configured to include heartwood formed at the inner center, sapwood that surrounds the heartwood, and an outer bark that surrounds the sapwood and forms the exterior. The portion from which the outer bark and the sapwood are removed from such a log is called the heartwood.

In the composition according to an aspect of the present disclosure, the pine heartwood extract is an extract of water, C₁-C₆ alcohol, or an aqueous C₁-C₆ alcohol solution. Here, C₁-C₆ alcohol may be methanol, ethanol, propanol, butanol, pentanol, or hexanol. Also, the aqueous C₁-C₆ alcohol solution may have a concentration of 5 to 99%. In a preferred embodiment, the extraction solvent is ethanol.

The pine heartwood extract of the present disclosure may be a solvent extract extracted with an extraction solvent, a fraction obtained by adding a fractionation solvent to an extract prepared by extraction with an extraction solvent, or a purified product obtained through chromatography of the fraction. The extraction solvent may be water, an organic solvent, or a mixed solvent thereof, which may be used for natural material extraction. The extraction solvent may be water, C₁-C₆ alcohol, or a mixture thereof, for example, water or ethanol. The pine heartwood extract of the present disclosure may be manufactured according to a typical plant extract manufacturing method. More specifically, the pine heartwood extract may be manufactured by adding an extraction solvent to ground dry pine heartwood from which impurities have been removed, followed by extraction. The extraction method using the solvent may be a cold extraction method, a warm extraction method, a solubilization extraction method, a reflux extraction method, or an ultrasonic extraction method.

As used herein, the term “fraction” refers to the result obtained by performing fractionation to separate a specific component or a group of specific components from a mixture containing various components.

Moreover, the fraction of the extract may be manufactured by adding a fractionation solvent to the extract prepared by the extraction method and then obtaining a fraction depending on the polarity of the fractionation solvent. The method of obtaining the fraction may be performed by layer separation or fractionation. More specifically, this method may be performed by adding fractionation solvents, for example, adding ethyl acetate and water in that order, to the extract, and then obtaining an ethyl acetate fraction and a water fraction that are layer separated. The fractionation method by layer separation may include sequentially adding solvents to the extract depending on the extent of non-polarity of the solvents and obtaining a fraction by layer separation for each application. For example, an ethyl acetate fraction resulting from adding ethyl acetate to an aqueous ethanol solution extract and fractionating an ethyl acetate layer by layer separation, and a water fraction, which is the layer remaining after separating the ethyl acetate fraction, may be obtained in that order. Chromatography for purification of the fraction may be performed using various chromatographic processes such as silica gel column chromatography, thin layer chromatography (TLC), high-performance liquid chromatography (HPLC), etc.

In the composition according to an aspect of the present disclosure, the concentration of the aqueous C₁-C₆ alcohol solution is 10% to 98% (v/v).

In the composition according to an aspect of the present disclosure, the pine heartwood extract is an ethanol extract.

In the composition according to an aspect of the present disclosure, the active ingredient promotes the activity of alcohol dehydrogenase (ADH).

In the composition according to an aspect of the present disclosure, the active ingredient promotes the activity of acetaldehyde dehydrogenase (ALDH).

Another aspect of the present disclosure provides a composition for preventing, ameliorating, or treating alcoholic liver disease, including a pine heartwood extract as an active ingredient.

The alcoholic liver disease may be at least one selected from among alcoholic fatty liver disease, alcoholic hepatitis, and alcoholic cirrhosis.

In the composition according to another aspect of the present disclosure, the pine heartwood extract is an extract of water, C₁-C₆ alcohol, or an aqueous C₁-C₆ alcohol solution.

In the composition according to another aspect of the present disclosure, the concentration of the aqueous C₁-C₆ alcohol solution is 10% to 98% (v/v).

In the composition according to another aspect of the present disclosure, the active ingredient promotes the activity of alcohol dehydrogenase (ADH).

In the composition according to another aspect of the present disclosure, the active ingredient promotes the activity of acetaldehyde dehydrogenase (ALDH).

In another aspect of the present disclosure, the composition is a functional health food.

The health food composition of the present disclosure contains a pine heartwood extract or a solvent fraction fractionated therefrom, and there is no particular limitation on types thereof. Examples of the food include drinks, meat, sausages, bread, biscuits, rice cakes, roasted and ground grains, chocolate, candies, snacks, confectionery, pizza, ramen, other noodles, gum, dairy products including ice cream, various soups, beverages, alcoholic beverages, vitamin complexes, dairy and processed milk products, etc. In addition thereto, the food includes all functional health foods in the typical sense. As an active ingredient, a pine heartwood extract or a solvent fraction fractionated therefrom may be added as is to food or used together with other foods or food ingredients, and may be used appropriately according to typical methods. The effective amount thereof may be appropriately determined depending on the purpose of use (prevention or amelioration) and may fall in the range of 0.001 to 70 wt % based on the total weight of the health food. However, for long-term intake for the purpose of health and hygiene or health control, the amount thereof may be equal to or less than the above lower limit, and if there is no problem in terms of safety, the active ingredient may be used in an amount equal to or greater than the above upper limit. For example, when manufacturing a health drink, in addition to the active ingredient, natural carbohydrates or flavoring agents may be contained as additives commonly used in beverage production. Examples of the natural carbohydrates may include typical sugars such as monosaccharides (e.g., glucose, fructose, etc.), disaccharides (e.g., maltose, sucrose, etc.), and polysaccharides (e.g., dextrin, cyclodextrin, etc.), and sugar alcohols such as xylitol, sorbitol, erythritol, etc. The natural carbohydrate may be contained in an amount of 1 to 20 wt %, preferably 5 to 10 wt %, based on the total weight of the health food. Examples of the flavoring agent may include natural flavoring agents (thaumatin, stevia extract, rebaudioside A, glycyrrhizin, etc.) and synthetic flavoring agents (saccharin, aspartame, etc.). Furthermore, various nutrients, vitamins, minerals (electrolytes), flavors (synthetic or natural flavors), colorants, pectic acid and salts thereof, alginic acid and salts thereof, organic acids, protective colloidal thickeners, pH adjusters, stabilizers, preservatives, glycerin, alcohol, carbonating agents used in carbonated drinks, etc. may be contained. Additionally, pulp for the production of natural fruit juice, fruit juice beverages, and vegetable beverages may be contained. There is no particular limitation on the amounts of these additives. The additive may be included in an amount of 0.1 to 20 wt % based on the total weight of the health food.

In another aspect of the present disclosure, the composition is a pharmaceutical composition.

Carriers, excipients, and diluents that may be contained in the pharmaceutical composition of the present disclosure may include at least one selected from among lactose, dextrose, sucrose, sorbitol, mannitol, xylitol, erythritol, maltitol, starch, gum acacia, alginate, gelatin, calcium phosphate, calcium silicate, cellulose, methyl cellulose, hydroxymethylcellulose, microcrystalline cellulose, silicified microcrystalline cellulose, povidone, crospovidone, croscarmellose sodium, polyvinylpyrrolidone, water, methylhydroxybenzoate, propylhydroxybenzoate, Neusilin, colloidal silicon dioxide, talc, magnesium stearate, colloidal magnesium stearate, and mineral oil.

The treatment amount of the pharmaceutical composition of the present disclosure may be determined by those skilled in the art in consideration of factors well known in the medical field, such as purpose of use, severity of disease, age, body weight, health, gender, sensitivity to the drug, treatment time, route, or type of material serving as an active ingredient. The composition may contain a pine heartwood extract or a fraction thereof as a single active ingredient. Specifically, the composition may not contain any single active ingredient other than a pine heartwood extract or a fraction thereof. In one embodiment, a method of treating psoriasis in a subject is provided, including treating the subject with the pharmaceutical composition in an amount effective to prevent or treat psoriasis. The subject may be a mammal. The mammal may be a human, dog, cat, cow, goat, or pig. Furthermore, the subject may be a human or an animal other than a human, and may be a human or a mammal other than a human. The treatment may be carried out through any common route so long as the composition may reach the desired organ. For example, the composition may be used through routes such as skin application, intraperitoneal administration, intravenous administration, intramuscular administration, subcutaneous administration, intradermal administration, transdermal patch, oral administration, intranasal administration, intrapulmonary administration, intrarectal administration, etc. Specifically, the composition may be used depending on the need through the route of application onto the skin, preferably the route of dermal application. The preferred dosage of the pharmaceutical composition of the present disclosure may vary depending on the patient's age, body weight, severity of disease, drug form, administration route and period, but may be appropriately determined by those skilled in the art. For desirable effects, the pharmaceutical composition of the present disclosure may be administered at 0.001 mg/kg to 1 mg/kg, preferably 0.1 mg/kg to 10 mg/kg per day. Administration may be performed several times a day at regular time intervals, preferably once to six times, depending on the judgment of the doctor or pharmacist.

Still another aspect of the present disclosure provides a kit including the pharmaceutical composition according to any one aspect of the present disclosure.

### MODE FOR INVENTION

A better understanding of the present disclosure may be obtained through the following examples. These examples are merely set forth to illustrate the present disclosure, and are not to be construed as limiting the scope of the present disclosure.

### Example 1. Obtaining Functional Material from Pine

Pine material grown in Yeongcheon, Gyeongsangbuk-do was purchased at the Gyeongdong medicinal herb market. The scientific name for the single variety of pine cultivated in Korea is Korean red pine. The active ingredient was obtained through the following experiment using the pine material.

### Example 1-1. 1. Extracting Functional Active Ingredient from Pine and Obtaining Extracted Powder (Extraction Method 1)

In order to extract pycnogenol and functional ingredients expected to be active ingredients of pine, 50 g of the pine material was used. Pine, particularly, pine needles (pine branches), Pinus densiflora roots (pine roots), and heartwood (pine trunk), were separately ground, added to 2 L of ethyl alcohol (95% ethanol), and subjected to ultrasonic extraction for 3 hours in a dark room.

In order to obtain large amounts of active ingredients, the extraction process was repeated three times, obtaining an extract of pycnogenol and functional ingredients in 6 L of ethyl alcohol.

In order to powder the extract, the extract was partially concentrated using a vacuum concentrator, a small amount of distilled water was added, and the extract was cooled to −80° C., after which a dry powder was obtained using a freeze dryer.

### Example 1-1. 2. Extracting Functional Active Ingredient from Pine and Obtaining Extracted Powder (Extraction Method 2)

In order to extract pycnogenol and functional ingredients expected to be active ingredients of pine, 50 g of pine (pine needles, Pinus densiflora roots, heartwood) was ground, added to 2 L of distilled water, and subjected to hot water extraction at 90-100° C. for 3 hours.

In order to obtain large amounts of active ingredients, the extraction process was repeated three times, obtaining an extract of pycnogenol and functional ingredients in 6 L of ethyl alcohol.

In order to powder the extract, the extract was partially concentrated using a vacuum concentrator and then cooled to −80° C., after which a dry powder was obtained using a freeze dryer.

### Example 2. Animal Experiment on Effect of Curing Hangover Using Pine Extract

### Example 2-1. Experimental Animals and Pretreatment Procedures

An animal experiment was conducted by dividing C57BL/6J mice weighing about 25-30 g into a total of 21 groups depending on the sample and the blood collection time after sample administration. Feeding of experimental mice was stopped at least 12 hours before sample administration. Thereafter, 300 mg/kg of the sample (pine needle H₂O extract, pine needle 95% EtOH extract, Pinus densiflora root H₂O extract, Pinus densiflora root 95% EtOH extract, heartwood EtOH extract) was orally administered to the experimental mice, and 30 minutes later, 20% EtOH (3 g/kg) was administered intraperitoneally. Here, the sample was dissolved in 1% dimethyl sulfoxide (DMSO) and then diluted with a 0.5% sodium carboxymethyl cellulose (CMC) solution before use.

### Example 2-2. Experimental Treatment Group

The experimental treatment groups were divided into a total of 7 groups, including untreated group (negative control), EtOH (positive control), EtOH+sample (300 mg/kg of pine needle H₂O extract), EtOH+sample (300 mg/kg of pine needle 95% EtOH extract), EtOH+sample (300 mg/kg of Pinus densiflora root H₂O extract), EtOH+sample (300 mg/kg of Pinus densiflora root 95% EtOH extract), and EtOH+sample (300 mg/kg of heartwood EtOH extract). For the number of repetitions, 12 mice (n=12) per experimental treatment group were treated and heart blood was collected from 4 mice each after 1, 3, and 5 hours.

### Example 2-3. Analysis of Ethanol Changes in Blood

Changes in the amount of ethanol in blood collected from the mouse heart were confirmed 1, 3, and 5 hours after alcohol administration. The collected blood was suspended 1:1 in 4 M perchloric acid (HClO₄) and then centrifuged at 3,000 rpm for 15 minutes to precipitate the protein. To neutralize the precipitated protein, 2 M calcium hydroxide (KOH) was added to adjust the pH to 6.5-7.5, followed by centrifugation at 12,000 rpm for 15 minutes, and the supernatant was used. Here, the concentration of ethanol was measured using an ethanol assay kit (Sigma-Aldrich).

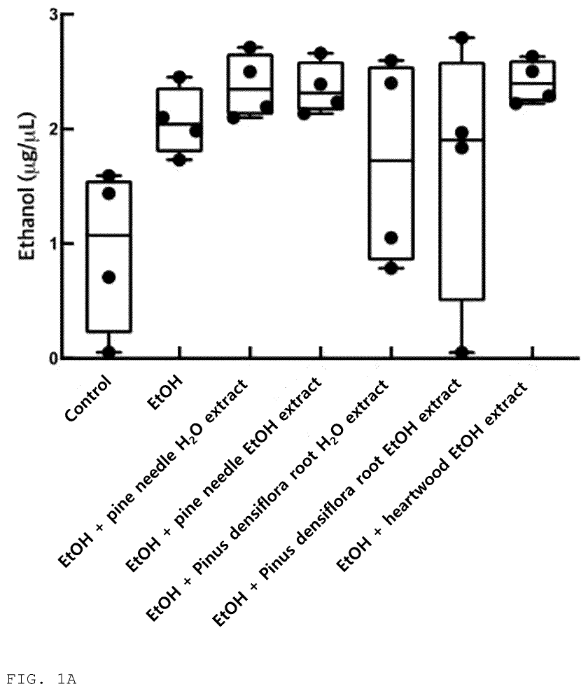

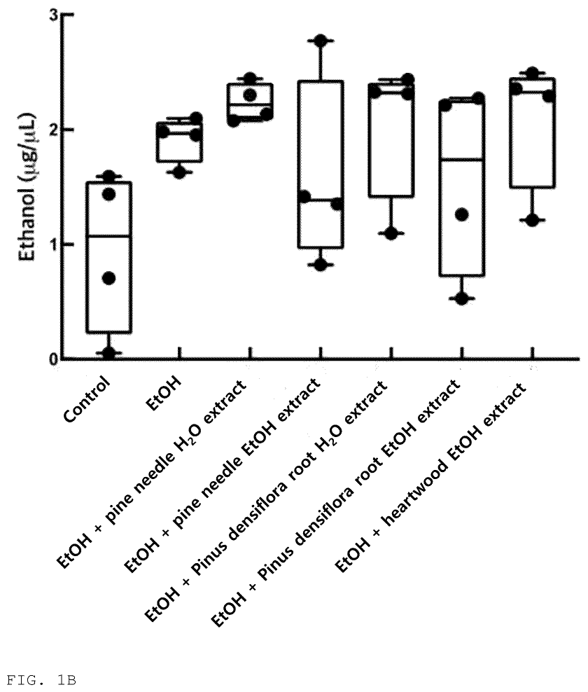

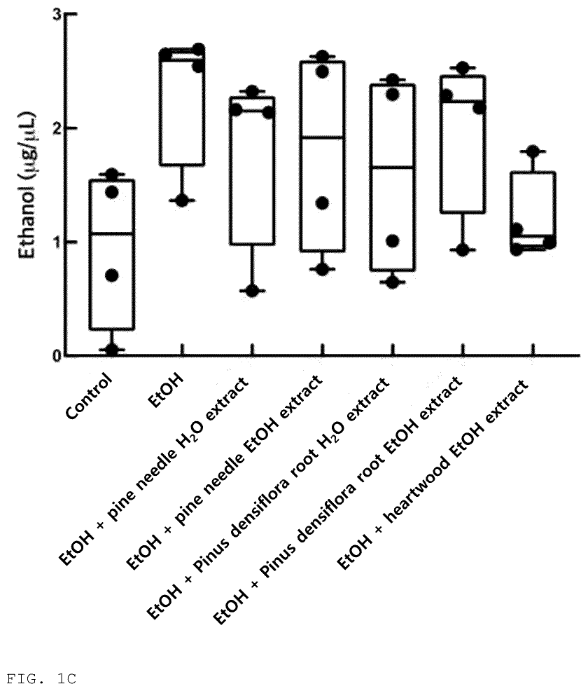

The experimental results are shown in FIGS. 1A to 1C. FIG. 1A shows the results after 1 hour, FIG. 1B shows the results after 3 hours, and FIG. 1C shows the results after 5 hours.

Based on the experimental results, it was confirmed that the pine heartwood extract increased activity of the alcohol decomposition enzyme, rapidly reducing the blood alcohol concentration after 3 hours.

### Example 2-4. Analysis of Changes in Activity of Alcohol Dehydrogenase (ADH)

Changes in the activity of alcohol dehydrogenase (ADH), which is an enzyme that converts ethanol into acetaldehyde in the liver, were confirmed. The liver obtained from mice was homogenized in 200 μl of ADH assay buffer and then centrifuged at 13,000 rpm for 10 minutes, after which the supernatant was measured using an alcohol dehydrogenase activity assay kit (Sigma-Aldrich).

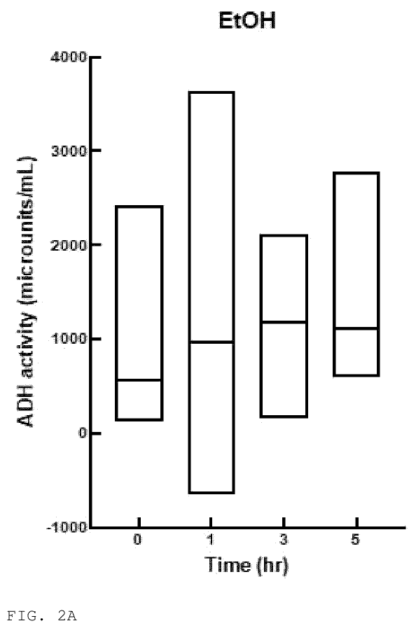

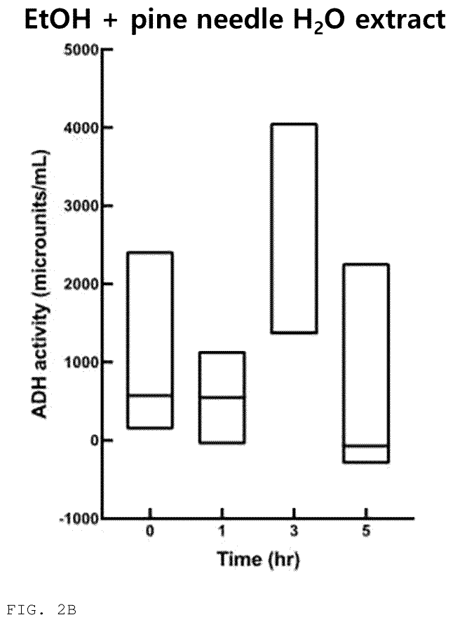

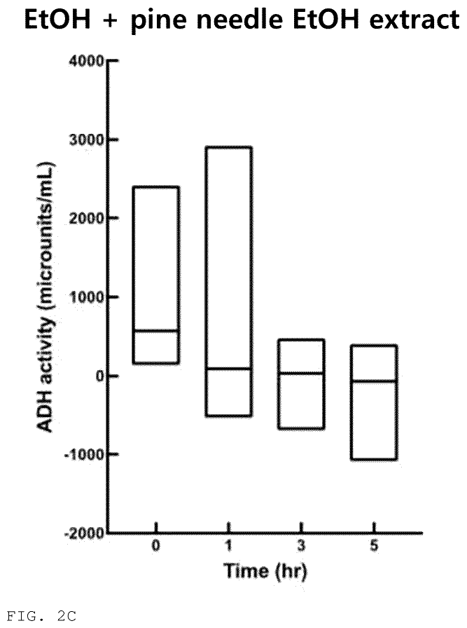

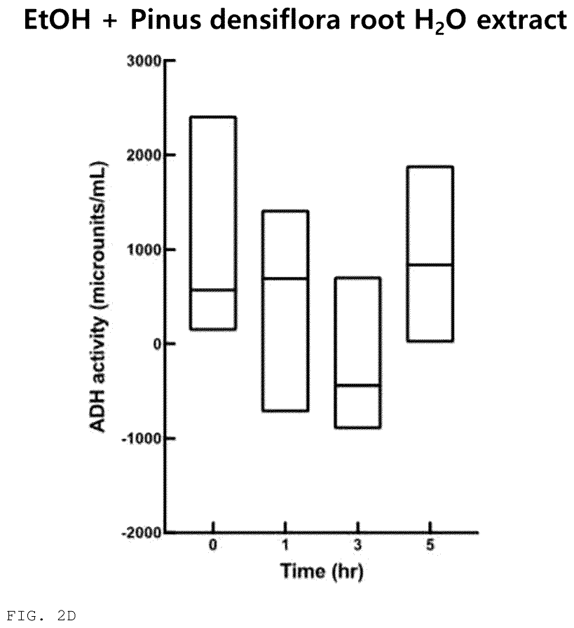

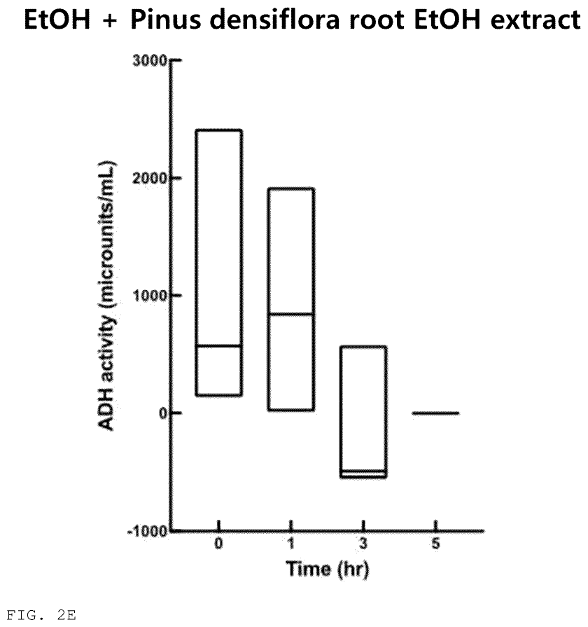

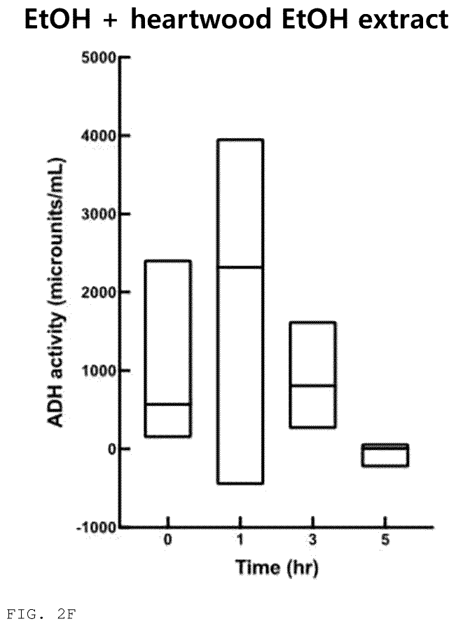

The experimental results are shown in FIGS. 2A to 2F. Based on the experimental results, it was confirmed that the heartwood extract increased activity of the alcohol decomposition enzyme initially compared to the other experimental groups. Therefore, the decomposition of acute alcohol hepatotoxicity was fast compared to the other experimental groups.

### Example 2-5. Analysis of Changes in Activity of Aldehyde Dehydrogenase (ALDH)

Changes in the activity of aldehyde dehydrogenase (ALDH), which is an enzyme that converts acetaldehyde into acetate, were confirmed using an aldehyde dehydrogenase activity colorimetric assay kit (Sigma-Aldrich). Mouse liver was homogenized in 200 μl of ALDH assay buffer and then centrifuged at 13,000 rpm for 10 minutes, after which the supernatant was measured as to enzyme activity.

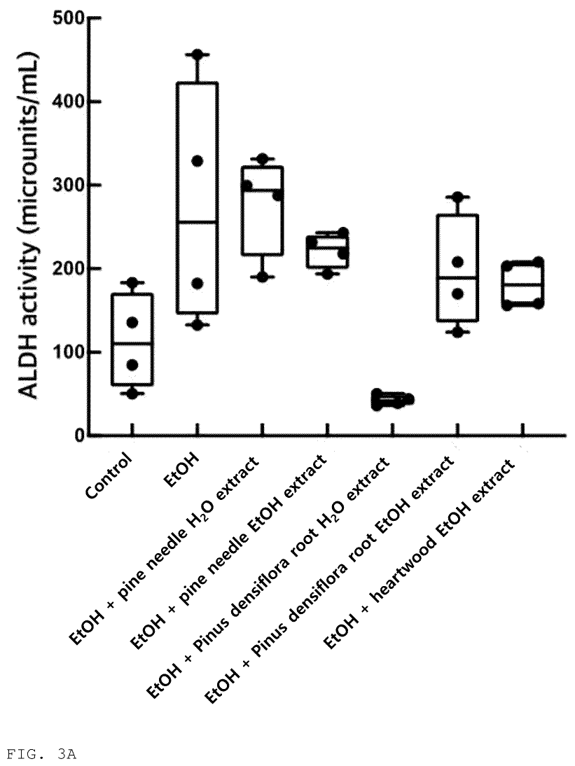

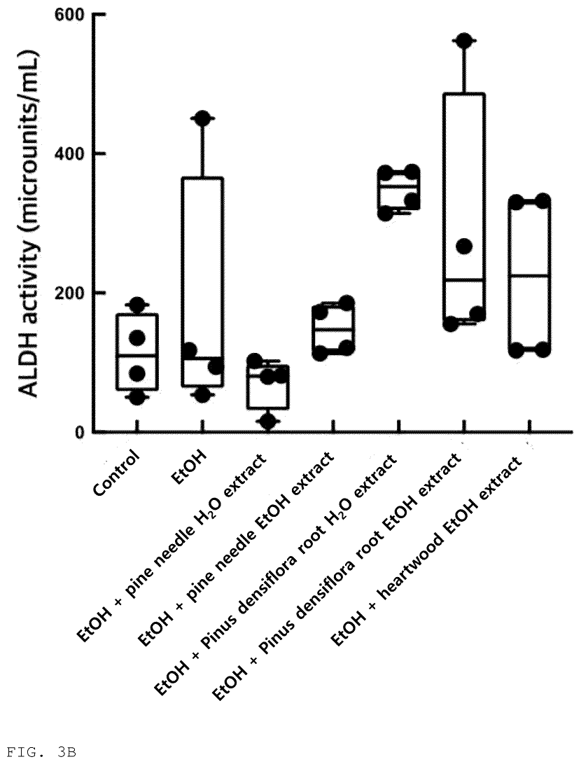

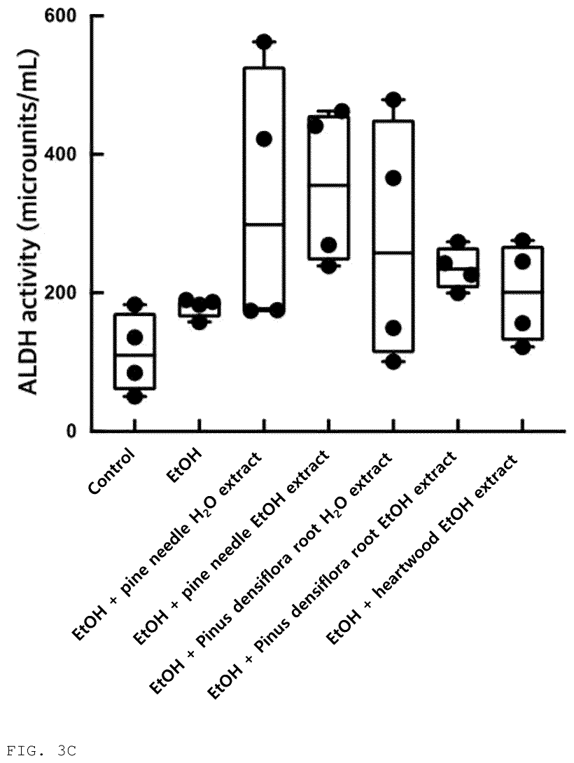

The experimental results are shown in FIGS. 3A to 3C. FIG. 3A shows the results after 1 hour, FIG. 3B shows the results after 3 hours, and FIG. 3C shows the results after 5 hours. Based on the experimental results, it was confirmed that the heartwood extract increased activity of the alcohol decomposition enzyme initially compared to the other experimental groups. Therefore, the decomposition of acute alcohol hepatotoxicity was fast compared to the other experimental groups.

Below, formulation examples of the composition according to the present disclosure are described, but are not to be construed as limiting the present disclosure and are merely set forth to provide a detailed description.

### [Formulation Example 1] Preparation of Pills

30 wt % of Example 1, 30 wt % of corn starch, 20 wt % of glycerin, and 20 wt % of sorbitol powder were mixed, and pills were prepared using a pill maker. The final weight of the contents was 3.5 g.

### [Formulation Example 2] Preparation of Tablets

30 wt % of the extract of Example 1, 20.5 wt % of lactose, 20 wt % of dextrin, 20 wt % of maltitol powder, and 7 wt % of xylitol powder were mixed, and granulated using a fluidized-bed dryer, and then 2.5 wt % of sugar ester was added, followed by tableting using a tablet press. The final weight of the contents was 2 g.

### [Formulation Example 3] Preparation of Granules

30 wt % of the extract of Example 1, 5 wt % of xylitol, and 65 wt % of isomalt were mixed, formed into granules using a fluidized-bed granulator, and then placed in pouches. The final weight of the contents was 2 g.

### [Formulation Example 4] Preparation of Injection

An injection was prepared by a typical method according to the composition shown in Table 1 below.

TABLE 1

| Mixing ingredients | Amount |
|---|---|
| Example 1 | 10-50 mg |
| Sterile distilled | Appropriate amount |
| water for injection | |
| pH adjuster | Appropriate amount |

[Formulation Example 5] Health food: A health food was prepared by a typical method according to the composition shown in Table 2 below.

TABLE 2

| Mixing ingredients | Amount | |
|---|---|---|
| Example 1 | 20 | mg |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |
| Ferrous sulfate | 1.75 | mg |
| Zinc oxide | 0.82 | mg |
| Magnesium carbonate | 25.3 | mg |
| Potassium phosphate monobasic | 15 | mg |
| Calcium phosphate dibasic | 55 | mg |
| Potassium citrate | 90 | mg |
| Calcium carbonate | 100 | mg |
| Magnesium chloride | 24.8 | mg |

[Formulation Example 5] Health drink: A health drink was prepared by a typical method according to the composition shown in Table 3 below.

TABLE 3

| Mixing ingredients | Amount | |
|---|---|---|
| Example 1 | 1000 | mg |
| Citric acid | 1000 | mg |
| Oligosaccharide | 100 | g |
| Taurine | 1 | g |

| | |
|---|---|
| Purified water | Balance |

Although specific embodiments of the present disclosure have been described with reference to the accompany drawings, those skilled in the art will appreciate that the present disclosure may be embodied in other specific forms without changing the technical spirit or essential features thereof. Thus, the embodiments described above should be understood to be non-limiting and illustrative in every way.

1. A composition for curing a hangover, comprising a pine heartwood extract as an active ingredient.

2. The composition of claim 1, wherein the pine heartwood extract is an extract of water, C₁-C₆ alcohol, or an aqueous C₁-C₆ alcohol solution.

3. The composition of claim 2, wherein a concentration of the aqueous C₁-C₆ alcohol solution is 10% to 98% (v/v).

4. The composition of claim 1, wherein the active ingredient promotes activity of alcohol dehydrogenase (ADH).

5. The composition of claim 1, wherein the active ingredient promotes activity of acetaldehyde dehydrogenase (ALDH).

6. A composition for preventing, ameliorating, or treating alcoholic liver disease, comprising a pine heartwood extract as an active ingredient.

7. The composition of claim 6, wherein the pine heartwood extract is an extract of water, C₁-C₆ alcohol, or an aqueous C₁-C₆ alcohol solution.

8. The composition of claim 7, wherein a concentration of the aqueous C₁-C₆ alcohol solution is 10% to 98% (v/v).

9. The composition of claim 6, wherein the active ingredient promotes activity of alcohol dehydrogenase (ADH).

10. The composition of claim 6, wherein the active ingredient promotes activity of acetaldehyde dehydrogenase (ALDH).

11. The composition of claim 6, wherein the composition is a functional health food.

12. The composition of claim 6, wherein the composition is a pharmaceutical composition.

13. The composition of claim 7, wherein the composition is a functional health food.

14. The composition of claim 8, wherein the composition is a functional health food.

15. The composition of claim 9, wherein the composition is a functional health food.

16. The composition of claim 10, wherein the composition is a functional health food.

17. The composition of claim 7, wherein the composition is a pharmaceutical composition.

18. The composition of claim 8, wherein the composition is a pharmaceutical composition.

19. The composition of claim 9, wherein the composition is a pharmaceutical composition.

20. The composition of claim 10, wherein the composition is a pharmaceutical composition.
